(19) United States
(12) Patent Application Publication
(10) Pub. No.: US 2004/0258642 A1
(43) Pub. Date: Dec. 23, 2004

(54) Cosmetic compositions

(76) Inventors: Joseph Frank Calello, Bridgewater, NJ (US); Anjali Abhimanyu Patil, Westfield, NJ (US); Robert Walter Sandewicz, Monroe Township, NJ (US)

Correspondence Address: Julie Blackburn, Revlon Consumer Products Corporation, Law Departmenrt, 237 Park Avenue, New York, NY, 10017, US

(21) Appl. No.: 10/860,168

(22) Filed: Jun. 3, 2004

Related U.S. Application Data
(60) Provisional application No. 60/479,558, filed on Jun. 18, 2003.

(51) Int. Cl. A61K 7/04; A61K 7/025

(52) U.S. Cl. 424/64

(57) ABSTRACT

Cosmetic compositions containing at least one organic compound having at least one short chain alkyl ether group bonded to at least one short chain alcohol; long wearing and/or transfer resistant cosmetic composition wherein at least a portion of the long wearing or transfer resistant property is provided by volatile silicones or volatile paraffinic hydrocarbons, the improvement wherein at least some of the volatile silicone or volatile paraffinic hydrocarbon is replaced with an organic solvent containing at least one short chain alkyl ether group bonded to at least one short chain alcohol or alcohol ester; and methods for improving the aesthetics of cosmetic compositions.

### RELATED APPLICATIONS

This application claims priority from provisional patent application Ser. No. 60/479,558, filed Jun. 18, 2003.

### TECHNICAL FIELD

The invention is in the field of cosmetic compositions for application to keratinous surfaces, and including but not limited to long wearing and/or transfer resistant cosmetic compositions.

### BACKGROUND OF THE INVENTION

Long wearing cosmetic compositions are becoming more and more desirable. Women today are very busy and do not have the time or inclination to frequently reapply makeup. Transfer resistant cosmetics are a sub-category of long wearing cosmetics, and are cosmetic products that exhibit particularly strong affinity for keratinous surfaces, generally resisting the tendency to blot off the surface while undergoing normal activities. Many long wearing, and most transfer resistant compositions contain volatile ingredients which are critical to achieving the long wearing benefit. In most transfer resistant cosmetic compositions the volatile oils are linear or cyclic silicones or paraffinic hydrocarbons. While these types of volatile silicones and hydrocarbons work very well in promoting transfer resistance in the compositions in which they are used, they do have certain drawbacks. For example, volatile paraffinic hydrocarbons can leave a greasy residue on skin or lips, and are not always the best solvents for the film forming polymers used in such cosmetics. Volatile silicones are also excellent in promoting the transfer resistant properties of cosmetics in which they are used, but their drawbacks include incompatibility with certain film forming polymers, and higher cost. In addition the very high flash points of some volatile silicones make them evaporate very quickly, which causes problems during the manufacturing step as well as in storage of the resulting product.

Accordingly, there is a need for different solvents for use in promoting the long wearing and/or transfer resistant properties of cosmetic compositions.

It is an object of the invention to provide long wearing and/or transfer resistant cosmetic compositions containing solvents other than volatile silicones or volatile paraffinic hydrocarbons that promote the long wearing and/or transfer resistant properties of the compositions.

It is also an object of the invention to provide a long wearing or transfer resistant cosmetic composition wherein a portion of the volatile silicone or paraffinic hydrocarbon normally present is substituted with a transfer resistant promoting solvent that is not a silicone or paraffinic hydrocarbon.

It is a further object of the invention to provide long wearing and/or transfer resistant compositions wherein the volatile component contributing to the transfer resistant and/or long wearing property comprises an ingredient containing at least one alkyl ether group bonded to a short chain alcohol or alcohol ester.

It is a further object of the invention to provide cosmetic compositions comprising at least one ingredient containing an alkyl ether group bonded to a short chain alcohol or alcohol ester.

### SUMMARY OF THE INVENTION

The invention comprises long wearing and/or transfer resistant cosmetic compositions wherein at least a portion of the long wearing or transfer resistant property is provided by volatile silicones or volatile paraffinic hydrocarbons, the improvement wherein at least some of the volatile silicone or volatile paraffinic hydrocarbon is replaced with an organic solvent containing at least one alkyl ether group bonded to at least one short chain alcohol or alcohol ester.

The invention further comprises a method for improving the wear, feel, application, and other aesthetic properties of long wearing cosmetic compositions wherein the long wearing property is achieved, at least in part, by use of volatile silicones or volatile paraffinic hydrocarbons, by substituting at least a portion of the volatile silicone or volatile paraffinic hydrocarbon with an organic solvent containing at least alkyl ether group bonded to at least one short chain alcohol or alcohol ester.

Another embodiment of the invention comprises a cosmetic composition, preferably a pigmented cosmetic composition, comprising at least one ingredient containing an alkyl ether group bonded to at least one short chain alcohol or alcohol ester.

### DETAILED DESCRIPTION

All percentages mentioned herein shall be percentages by weight unless otherwise indicated.

In one embodiment, the compositions of the invention are long wearing or transfer resistant cosmetic compositions wherein the long wearing or transfer resistant property of the composition is imparted, at least in part, by the presence of volatile silicones and/or volatile hydrocarbons. The compositions may be anhydrous or in the emulsion form, and may be lipsticks, foundation makeup, blush, eyeshadow, concealer, mascara, skin lotions, creams, and the like. The compositions may be in the liquid, solid, or semi-solid form. Most preferably the compositions are lipsticks.

Another embodiment of the invention is directed to cosmetic compositions, preferably pigmented cosmetic compositions, which comprise at least one ingredient containing an alkyl ether group bonded to at least one short chain alcohol or alcohol ester.

The term “transfer resistant” means that the composition, when applied to the keratinous surface, exhibits a reduced tendency to transfer from that surface onto another surface such as the back of the hand or a tissue when that other surface is placed into contact with the keratinous surface having the cosmetic composition applied thereto.

The term “long wearing” means that the composition, when applied to the keratinous surface, stays on the surface for a longer period of time.

The term “keratinous surface” means, in the context of this invention, skin (including lips), hair, or nails; most preferably skin and lips.

I. The Compositions of the Invention

The various types of ingredients that may be found in the cosmetic compositions of the invention are further described herein.

A. The Organic Component

In one embodiment of the cosmetic compositions of the invention, the volatile silicone or volatile paraffinic hydrocarbon that is present to contribute to the transfer resistant and/or long wearing properties is at least partially, possibly completely, replaced with an organic solvent comprised of one or more organic compounds containing at least one alkyl ether group bonded to at least one short chain alcohol or alcohol ester. Even though at least a portion of the volatile silicone or volatile paraffinic hydrocarbon normally present in the composition has been removed and substituted with the organic solvent, the composition still retains its transfer resistant and/or long wearing characteristics. In fact, the organic component is as effective as the volatile silicone or volatile paraffinic hydrocarbon in contributing to the long wearing or transfer resistant properties of the composition.

In another embodiment of the invention, the cosmetic composition comprises at least one or more organic compounds or solvents containing at least one alkyl ether group bonded to at least one short chain alcohol or alcohol ester. The composition may any type of cosmetic composition, and not necessarily long wearing or transfer resistant.

Preferably, the organic compound or solvent is volatile or near volatile. The term “volatile” means that the organic solvent has a vapor pressure of at least about 2 mm. of mercury at 20° C. The term “near volatile” means that the organic solvent has a vapor pressure ranging from about 1.0 to 2 mm of mercury at 20° C. The organic solvent may be present alone or in a mixture of compounds. Preferably the viscosity of the solvent or mixture ranges from about 0.1-12 centipoise, preferably about 0.3-8 centipoise, more preferably about 0.5-5 centipoise at room temperature (25° C.). The organic solvent is preferably present ranging from about 0.1-95%, preferably 0.5-75%, more preferably about 1-20% by weight of the total composition. In the preferred compositions in one embodiment of the invention, the organic solvent is substituted for about 1-50%, preferably about 5-40% of the volatile silicone or volatile paraffinic hydrocarbon.

The alkyl ether group is preferably a short chain alkyl ether group. The term “short chain” means an alkyl ether group having 1, 2, 3, 4, 5, or 6 carbon atoms. Preferably, the alkyl ether group is a C₁₋₄, more preferably a C₁₋₃ alkyl ether group.

The alkyl ether group is bonded to at least one short chain alcohol or alcohol ester. The short chain alcohol may be a mono-, di-, or polyhydric alcohol. The term “short chain” when used in conjunction with “alcohol” means an alcohol having 1, 2, 3, 4, 5, or 6 carbon atoms. Examples of short chain alcohols are methanol, ethanol, propanol, butanol, pentanol, hexanol, which may be substituted with one or more hydroxyl groups.

Suitable alcohol esters are short chain alcohols (as defined above) that are esterified with carboxylic acids having 1, 2, 3, 4, 5, or 6 carbon atoms. The carboxylic acids may also be substituted with one or more hydroxyl groups.

A wide variety of organic compounds suitable for use in place of the volatile silicone or volatile paraffinic hydrocarbons, or suitable for use in the cosmetic compositions of the invention in general whether long wearing or not, can be identified by simply combining one or more such alkyl ether groups with one or more of such short chain alcohols or alcohol esters.

Particularly preferred are organic compounds such as propylene glycol ethyl ether acetate, propylene glycol butyl ether, propylene glycol methyl ether acetate, propylene glycol methyl ether, methoxypropanol, or mixtures thereof.

B. Volatile Silicones or Volatile Paraffinic Hydroccarbons

In one embodiment of the invention, the organic compound is used to replace at least some, possibly all of the volatile silicones or volatile paraffinic hydrocarbons that may be present in the composition. In the event the organic compound is used to partially replace the volatile silicones or paraffinic hydrocarbons present, the composition may contain such materials. Examples of suitable volatile silicones or paraffinic hydrocarbons that may be present include:

1. Volatile Silicones

Cyclic silicones are of the general formula:

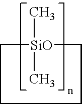

where n=3-6.

Linear volatile silicones in accordance with the invention have the general formula:

(CH₃)₃Si—O—[Si(CH₃)₂—O]ₙ—Si(CH₃)₃

where n=0, 1, 2, 3, 4, 5, 6, or 7, preferably 0, 1, 2, 3, or 4.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids include octamethylcyclotetrasiloxane, octamethyltrisiloxane, decamethyltetrasiloxane, decamethylcyclopentasiloxane, hexamethyldisiloxane, and mixtures thereof.

(b). Paraffinic Hydrocarbons

Volatile paraffinic hydrocarbons that may be present include various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260 degrees C., and a viscosity of less than 10 centipoise at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable. Examples of suitable volatile paraffinic hydrocarbons include isohexadecane, isododecane, or mixtures thereof.

C. Pigments and Powders

The composition may also comprise a variety of particulates including pigments, powders, and mixtures thereof. Suggested ranges of such ingredients are about 0.05-70%, preferably about 0.1-25%, more preferably about 0.5-20% by weight of the total composition.

Suitable pigments may be organic or inorganic. Examples of organic pigments include red, green, blue, yellow, violet, orange, and mixtures thereof. Also suitable are Lakes of such pigments, which means that the organic pigments are reacted with a metal salt such as calcium, aluminum, barium, zirconium, and the like to form salts. Preferred are aluminum Lakes of the organic pigments, which is where the organic pigment is reacted with aluminum to form the aluminum salt. Formation of the metal salt of the organic pigment will generally also convert the pigment from a water soluble pigment into a water insoluble pigment, and such pigments tend to become lipophilic in nature, meaning that they will exhibit affinity for lipophilic or oily ingredients in the composition. Examples of organic pigment families that may be used herein include azo, (including monoazo and diazo), fluoran, xanthene, indigoid, triphenylmethane, anthroquinone, pyrene, pyrazole, quinoline, quinoline, or metallic salts thereof. Preferred are D&C colors, FD&C colors, or Lakes of D&C or FD&C colors. The term “D&C” means drug and cosmetic colors that are approved for use in drugs and cosmetics by the FDA. The term “FD&C” means food, drug, and cosmetic colors that are approved for use in foods, drugs, and cosmetics by the FDA. Certified D&C and FD&C colors are listed in 21 CFR 74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable Lakes of D&C and FD&C colors are defined in 21 CFR 82.51. Particularly preferred are Lakes formed by the reaction of the organic pigment with a metallic salt such as aluminum, calcium, zirconium, barium, and the like. Suitable reds include pigments from the monoazo, disazo, fluoran, xanthene, or indigoid families or Lakes thereof, such as Red 4, 6, 7, 17, 21, 22, 27, 28, 30, 31, 33, 34, 36, and Red 40. Also suitable are Lakes of such red pigments. Typically the metal salts are aluminum, barium, and the like. Most preferred are Aluminum Lakes of the various red pigments mentioned herein.

Suitable yellows include wherein the yellow pigment is a pyrazole, monoazo, fluoran, xanthene, quinoline, or salt thereof. Suitable yellows include Yellow 5, 6, 7, 8, 10, and 11, as well as Lakes of such yellow pigments.

Suitable violets include those from the anthroquinone family, such as Violet 2 and Lakes thereof. Examples of orange pigments are Orange 4, 5, 10, 11, or Lakes thereof.

Also suitable are inorganic pigments that include iron oxides such as red, blue, black, green, and yellow; titanium dioxide, bismuth oxychloride, and the like. Preferred are iron oxides.

It may also be desirable to include one or more particulate fillers in the claimed composition. If so, suggested ranges are about 0.001-40%, preferably about 0.05-35%, more preferably about 0.1-30% by weight of the total composition. Preferably, the particulate fillers include non-pigmentitious particles that generally have a particle size ranging from about 0.02 to 200, preferably 0.5 to 100, microns. Suitable particle fillers include titanated mica, fumed silica, spherical silica, polymethylmethacrylate, micronized teflon, boron nitride, mica, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silk powder, silica, talc, mica, zinc laurate, zinc myristate, zinc rosinate, alumina, attapulgite, calcium carbonate, calcium silicate, dextran, kaolin, nylon, silica silylate, sericite, soy flour, tin oxide, titanium hydroxide, trimagnesium phosphate, walnut shell powder, or mixtures thereof. The particulates may also be in the fiber form, such as cellulose fibers, rayon fibers, nylon or silk fibers and the like. Such fibers are generally circular in cross-section and have a discernable length. Preferably the length ranges from 1 to 5 mm.

The above mentioned pigments, powders or fibers may be inherently lipophilic or hydrophilic. The term “lipophilic” when used in this context means that the particulates will exhibit an affinity for lipophilic ingredients, or will be soluble or dispersible in lipophilic ingredients such as nonpolar oils. The term “hydrophilic” when used in this context means that the particulates exhibit an affinity for, or are soluble or dispersible in, water. For example, many types of organic pigments are hydrophilic and will be soluble or dispersible in water. On the other hand, in some cases, if the organic pigments are reacted with metal salts to form Lakes, in some cases the pigments will then exhibit a more lipophilic character. In the compositions of the invention, the pigments may be inherently lipophilic or inherently hydrophilic, depending on the properties desired. Further, if the pigments, particles or fibers are hydrophilic or if it is desired to increase their already lipophilic character, it may be desired to further surface treat the particulates with lecithin, amino acids, mineral oil, silicone oil or various other agents either alone or in combination, which coat the particulate surface and render the particles more lipophilic in nature. The term “lipophilic” means that the pigment or particles will be compatible with the lipophilic or oily phase of the composition. In the case of an emulsion, a lipophilic particle will have an affinity for the oily phase of the emulsion.

In the most preferred embodiment of the invention the composition comprises a mixture of organic or inorganic pigments in combination with non-pigmentitious particulates. Such pigments may be hydrophilic or lipophilic in character.

D. Film Forming Polymers

The composition may also comprise one or more film forming polymers. If so, suggested ranges are from about 0.01-90%, preferably about 0.5-80%, more preferably about 1-70% by weight of the total composition. A variety of film forming polymers are suitable including silicones, copolymers of silicone and organic moieties, and various types of organic synthetic polymers. Examples of such polymers are further described below.

1. Silicone Film Forming Polymers

(a) Siloxane Polymeric Resins and Gums

Siloxane polymeric resins that comprises tetrafunctional or trifunctional units either alone or in combination with monofunctional units are suitable silicone film forming polymers. The term “siloxane polymeric resin” means that the siloxane is a polymer, or is comprised of repeating units or “mers”.

The term “resin” means that the siloxane polymer provides substantive, resinous, film forming properties when applied to skin. In the context of this invention, the term “resin” will mean a siloxane containing enough cross-linking to provide substantive, film forming properties. The term cross-linking means a moiety where the silicon atom is bonded to at least three, preferably four oxygen atoms when the moiety is polymerized with another siloxane unit.

The term “film forming” means that the siloxane resin is capable of forming a film, in particular, a substantive film, on the keratinous surface to which it is applied.

The term monofunctional unit means a siloxy unit that contains one silicon atom bonded to one oxygen atom, with the remaining three substituents on the silicon atom being other than oxygen. In particular, in a monofunctional siloxy unit, the oxygen atom present is shared by 2 silicon atoms when the monofunctional unit is polymerized with one or more of the other units. In silicone nomenclature used by those skilled in the art, a monofunctional siloxy unit is designated by the letter “M”, and means a unit having the general formula:

R₁R₂R₃SiO_{l/2}

wherein R₁, R₂, and R₃ are each independently C₁₋₃₀, preferably C₁₋₁₀, more preferably C₁₋₄ straight or branched chain alkyl, which may be substituted with phenyl or one or more hydroxyl groups; phenyl; alkoxy (preferably C₁₋₂₂, more preferably C₁₋₆); or hydrogen. The SiO_{1/2} designation means that the oxygen atom in the monofunctional unit is bonded to, or shared, with another silicon atom when the monofunctional unit is polymerized with one or more of the other types of units. For example, when R₁, R₂, and R₃ are methyl the resulting monofunctional unit is of the formula:

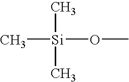

When this monofunctional unit is polymerized with one or more of the other units the oxygen atom will be shared by another silicon atom, i.e. the silicon atom in the monofunctional unit is bonded to ½ of this oxygen atom.

The term “difunctional siloxy unit” is generally designated by the letter “D” in standard silicone nomenclature. If the D unit is substituted with substituents other than methyl the “D”designation is sometimes used, which indicates a substituent other than methyl. For purposes of this disclosure, a “D” unit has the general formula:

R₁R₂SiO_{2/2}

wherein R₁ and R₂ are defined as above. The SiO_{2/2} designation means that the silicon atom in the difunctional unit is bonded to two oxygen atoms when the unit is polymerized with one or more of the other units. For example, when R₁, R₂, are methyl the resulting difunctional unit is of the formula:

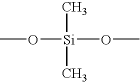

When this difunctional unit is polymerized with one or more of the other units the silicon atom will be bonded to two oxygen atoms, i.e. will share two one-halves of an oxygen atom.

The term “trifunctional siloxy unit” is generally designated by the letter “T” in standard silicone nomenclature. A “T” unit has the general formula:

R₁SiO_{3/2}

wherein R₁ is as defined above. The SiO_{3/2} designation means that the silicon atom is bonded to three oxygen atoms when the unit is copolymerized with one or more of the other units. For example when R₁ is methyl the resulting trifunctional unit is of the formula:

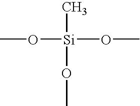

When this trifunctional unit is polymerized with one or more of the other units, the silicon atom shares three oxygen atoms with other silicon atoms, i.e. will share three halves of an oxygen atom.

The term “tetrafunctional siloxy unit” is generally designated by the letter “Q” in standard silicone nomenclature. A “Q” unit has the general formula:

SiO_{4/2}

The SiO_{4/2} designation means that the silicon shares four oxygen atoms (i.e. four halves) with other silicon atoms when the tetrafunctional unit is polymerized with one or more of the other units. The SiO_{4/2} unit is best depicted as follows:

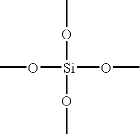

The film forming siloxane resins that may be used in the compositions of the invention comprises D, T or Q units either alone or in combination with M units. In addition, there may be one or more of the other types of units present in the polymer.

The film forming polymeric siloxane resin may be a liquid, semi-solid, or solid at room temperature.

Typically T or MT silicones are referred to as silsesquioxanes, and in the case where M units are present methylsilsesquioxanes. Preferred are T silicones having the following general formula:

(R₁SiO_{3/2})x

where x ranges from about 1 to 100,000, preferably about 1-50,000, more preferably about 1-10,000, and wherein R₁ is as defined above. Such MT silicones are generally referred to as polymethylsilsesquioxane which are silsesquioxanes containing methyl groups.

Examples of specific polysilsesquioxanes that may be used are manufactured by Wacker Chemie under the Resin MK designation. This polysilsesquioxane is a polymer comprise of T units and, optionally one or more D (preferably dimethylsiloxy) units. This particularly polymer may have ends capped with ethoxy groups, and/or hydroxyl groups, which may be due to how the polymers are made, e.g. condensation in aqueous or alcoholic media. Other suitable polysilsesquioxanes that may be used as the film forming polymer include those manufactured by Shin-Etsu Silicones and include the “KR” series, e.g. KR-220L, 242A, and so on. These particular silicone resins may contain endcap units that are hydroxyl or alkoxy groups which may be present due to the manner in which such resins are manufactured.

Also suitable are MQ resins, which are siloxy silicate polymers having the following general formula:

[(RR′R″)₃SiO_{1/2}]ₓ[SiO₂]_{y}

wherein R, R′ and R″ are each independently a C₁₋₁₀ straight or branched chain alkyl or phenyl, and x and y are such that the ratio of (RR′R″)₃SiO_{1/2} units to SiO₂ units ranges from about 0.5 to 1 to 1.5 to 1. Preferably R, R′ and R″ are a C₁₋₆ alkyl, and more preferably are methyl and x and y are such that the ratio of (CH₃)₃SiO_{1/2} units to SiO₂ units is about 0.75 to 1. Most preferred is this trimethylsiloxysilicate containing 2.4 to 2.9 weight percent hydroxyl groups which is formed by the reaction of the sodium salt of silicic acid, chlorotrimethylsilane, and isopropyl alcohol. The manufacture of trimethylsiloxysilicate is set forth in U.S. Pat. Nos. 2,676,182; 3,541,205; and 3,836,437, all of which are hereby incorporated by reference. Trimethylsiloxysilicate as described is available from GE Silicones under the tradename SR-1000, which is a solid particulate material. Also suitable is Dow Corning 749 which is a mixture of volatile cyclic silicone and trimethylsiloxysilicate.

The film forming siloxane polymeric resins that may be used in the composition are made according to processes well known in the art. In general siloxane polymers are obtained by hydrolysis of silane monomers, preferably chlorosilanes. The chlorosilanes are hydrolyzed to silanols and then condensed to form siloxanes. For example, Q units are often made by hydrolyzing tetrachlorosilanes in aqueous or aqueous/alcoholic media to form the following:

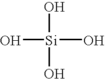

The above hydroxy substituted silane is then condensed or polymerized with other types of silanol substituted units such as:

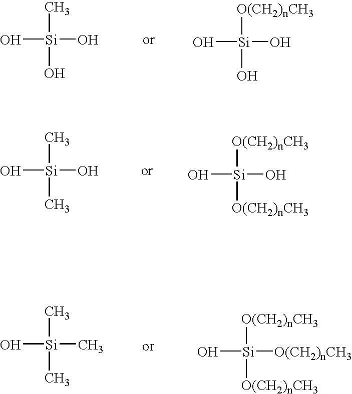

wherein n is 0-10, preferably 0-4.

Because the hydrolysis and condensation may take place in aqueous or aqueous/alcoholic media wherein the alcohols are preferably lower alkanols such as ethanol, propanol, or isopropanol, the units may have residual hydroxyl or alkoxy functionality as depicted above. Preferably, the resins are made by hydrolysis and condensation in aqueous/alcoholic media, which provides resins that have residual silanol and alkoxy functionality. In the case where the alcohol is ethanol, the result is a resin that has residual hydroxy or ethoxy functionality on the siloxane polymer. The silicone film forming polymers used in the compositions of the invention are generally made in accordance with the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which is hereby incorporated by reference in its entirety.

Also suitable are linear, high molecular weight silicones that are semi-solids, solids, or gums at room temperature. Examples of such silicones include dimethicones having viscosities ranging from about 100,000 to 10 million, or 500,000 to 10 million centipoise or dimethicone copolyols having the same viscosity range.

Also suitable are silicone esters as disclosed in U.S. Pat. No. 4,725,658 and U.S. Pat. No. 5,334,737, which are hereby incorporated by reference. Such silicone esters comprise units of the general formula RₐR^{E}_{b}SiO_{[4-(a+b)/2]} or R¹³ₓR^{E}_{y}SiO_{l/2}, wherein R and R¹³ are each independently an organic radical such as alkyl, cycloalkyl, or aryl, or, for example, methyl, ethyl, propyl, hexyl, octyl, decyl, aryl, cyclohexyl, and the like, a is a number ranging from 0 to 3, b is a number ranging from 0 to 3, a+b is a number ranging from 1 to 3, x is a number from 0 to 3, y is a number from 0 to 3 and the sum of x+y is 3, and wherein R^{E} is a carboxylic ester containing radical. Preferred R_{E} radicals are those wherein the ester group is formed of one or more fatty acid moieities (e.g. of about 2, often about 3 to 10 carbon atoms) and one or more aliphatic alcohol moieities (e.g. of about 10 to 30 carbon atoms). Examples of such acid moieties include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxy)propane. Preferably the ester subgroup (i.e. the carbonyloxy radical) will be linked to the silicon atom by a divalent aliphatic chain that is at least 2 or 3 carbon atoms in length, e.g. an alkylene group or a divalent alkyl ether group. Most preferably that chain will be part of the alcohol moiety, not the acid moiety. Such silicones may be liquids or solids at room temperature.

(b). Copolymers of Silicone and Ethylenically Unsaturated Monomers

Another type of film forming polymer that may be used in the compositions of the invention is obtained by reacting silicone moieties with ethylenically unsaturated monomers. The resulting copolymers may be graft or block copolymers. The term “graft copolymer” is familiar to one of ordinary skill in polymer science and is used herein to describe the copolymers which result by adding or “grafting” polymeric side chain moieties (i.e. “grafts”) onto another polymeric moiety referred to as the “backbone”. The backbone may have a higher molecular weight than the grafts. Thus, graft copolymers can be described as polymers having pendant polymeric side chains, and which are formed from the “grafting” or incorporation of polymeric side chains onto or into a polymer backbone. The polymer backbone can be a homopolymer or a copolymer. The graft copolymers are derived from a variety of monomer units.

One type of polymer that may be used as the film forming polymer is a vinyl-silicone graft or block copolymer having the formula:

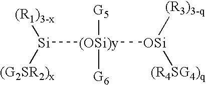

wherein G₅ represents monovalent moieties which can independently be the same or different selected from the group consisting of alkyl, aryl, aralkyl, alkoxy, alkylamino, fluoroalkyl, hydrogen, and -ZSA; A represents a vinyl polymeric segment consisting essentially of a polymerized free radically polymerizable monomer, and Z is a divalent linking group such as C₁₋₁₀ alkylene, aralkylene, arylene, and alkoxylalkylene, most preferably Z methylene or propylene.

G₆ is a monovalent moiety which can independently be the same or different selected from the group consisting of alkyl, aryl, aralkyl, alkoxy, alkylamino, fluoroalkyl, hydrogen, and -ZSA;

G₂ comprises A;

G₄ comprises A;

R₁ is a monovalent moiety which can independently be the same or different and is selected from the group consisting of alkyl, aryl, aralkyl, alkoxy, alkylamino, fluoroalkyl, hydrogen, and hydroxyl; but preferably C₁₋₄ alkyl or hydroxyl, and most preferably methyl.

R₂ is independently the same or different and is a divalent linking group such as C₁₋₁₀ alkylene, arylene, aralkylene, and alkoxyalkylene, preferably C₁₋₃ alkylene or C₇₋₁₀ aralkylene, and most preferably —CH₂— or 1,3-propylene, and

R₃ is a monovalent moiety which is independently alkyl, aryl, aralkyl, alkoxy, alkylamino, fluoroalkyl, hydrogen, or hydroxyl, preferably C₁₋₄ alkyl or hydroxyl, most preferably methyl;

R₄ is independently the same or different and is a divalent linking group such as C₁₋₁₀ alkylene, arylene, aralkylene, alkoxyalkylene, but preferably C₁₋₃ alkylene and C₇₋₁₀ alkarylene, most preferably —CH₂— or 1,3-propylene.

x is an integer of 0-3;

y is an integer of 5 or greater; preferably 10 to 270, and more preferably 40-270; and

q is an integer of 0-3.

These polymers are described in U.S. Pat. No. 5,468,477, which is hereby incorporated by reference. Most preferred is poly(dimethylsiloxane)-g-poly(isobutyl methacrylate), which is manufactured by 3-M Company under the tradename VS 70 IBM. This polymer may be purchased in the dry particulate form, or as a solution where the polymer is dissolved in one or more solvents such as isododecane. Preferred is where the polymer is in dry particulate form, and as such it can be dissolved in one or more of the liquids comprising the liquid carrier. This polymer has the CTFA name Polysilicone-6.

Another type of such a polymer comprises a vinyl, methacrylic, or acrylic backbone with pendant siloxane groups and pendant fluorochemical groups. Such polymers preferably comprise comprise repeating A, C, D and optionally B monomers wherein:

A is at least one free radically polymerizable acrylic or methacrylic ester of a 1,1,-dihydroperfluoroalkanol or analog thereof, omega-hydridofluoroalkanols, fluoroalkylsulfonamido alcohols, cyclic fluoroalkyl alcohols, and fluoroether alcohols,

B is at least one reinforcing monomer copolymerizable with A,

C is a monomer having the general formula X(Y)nSi(R)3-m Z.m wherein

X is a vinyl group copolymerizable with the A and B monomers,

Y is a divalent linking group which is alkylene, arylene, alkarylene, and aralkylene of 1 to 30 carbon atoms which may incorporate ester, amide, urethane, or urea groups,

n is zero or 1;

m is an integer of from 1 to 3,

R is hydrogen, C₁₋₄ alkyl, aryl, or alkoxy,

Z is a monovalent siloxane polymeric moiety; and

D is at least one free radically polymerizable acrylate or methacrylate copolymer.

Such polymers and their manufacture are disclosed in U.S. Pat. Nos. 5,209,924 and 4,972,037, which are hereby incorporated by reference. More specifically, the preferred polymer is a combination of A, C, and D monomers wherein A is a polymerizable acrylic or methacrylic ester of a fluoroalkylsulfonamido alcohol, and where D is a methacrylic acid ester of a C₁₋₂ straight or branched chain alcohol, and C is as defined above. Most preferred is a polymer having moieties of the general formula:

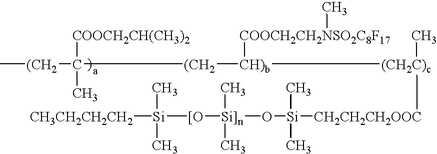

wherein each of a, b, and c has a value in the range of 1-100,000, and the terminal groups are selected from the group consisting of a C₁₋₂₀ straight or branched chain alkyl, aryl, and alkoxy and the like. These polymers may be purchased from Minnesota Mining and Manufacturing Company under the tradenames “Silicone Plus” polymers. Most preferred is poly(isobutyl methacrylate -co-methyl FOSEA)-g-poly(dimethylsiloxane) which is sold under the tradename SA 70-5 IBMMF.

Another suitable silicone acrylate copolymer is a polymer having a vinyl, methacrylic, or acrylic polymeric backbone with pendant siloxane groups. Such polymers as disclosed in U.S. Pat. Nos. 4,693,935, 4,981,903, 4,981,902, and which are hereby incorporated by reference. Preferably, these polymers are comprised of A, C, and optionally B monomers wherein:

A is at least on free radically polymerizable vinyl, methacrylate, or acrylate monomer;

B, when present, is at least one reinforcing monomer copolymerizable with A,

C is a monomer having the general formula:

X(Y)ₙSi(R)₃₋ₘZₘ

wherein:

X is a vinyl group copolymerizable with the A and B monomers;

Y is a divalent linking group;

n is zero or 1;

m is an integer of from 1 to 3;

R is hydrogen, C₁₋₁₀ alkyl, substituted or unsubstituted phenyl, Cₗ₋₁₀ alkoxy; and

Z is a monovalent siloxane polymeric moiety.

Examples of A monomers are lower to intermediate methacrylic acid esters of C₁₋₁₂ straight or branched chain alcohols, styrene, vinyl esters, vinyl chloride, vinylidene chloride, acryloyl monomers, and so on.

The B monomer, if present, is a polar acrylic or methacrylic monomer having at least one hydroxyl, amino, or ionic group (such as quaternary ammonium, carboxylate salt, sulfonic acid salt, and so on).

The C monomer is as above defined.

A particularly effective film forming polymer in this category is Polysilicone-6, which is a dry particulate material that may be used as is or solubilized in one or more ingredients that form the liquid carrier.

Examples of other suitable copolymers that may be used herein, and their method of manufacture, are described in detail in U.S. Pat. No. 4,693,935, Mazurek, U.S. Pat. No. 4,728,571, and Clemens et al., both of which are incorporated herein by reference. Additional grafted polymers are also disclosed in EPO Application 90307528.1, published as EPO Application 0 408 311, U.S. Pat. No. 5,061,481, Suzuki et al., U.S. Pat. No. 5,106,609, Bolich et al., U.S. Pat. No. 5,100,658, Bolich et al., U.S. Pat. No. 5,100,657, Ansher-Jackson, et al., U.S. Pat. No. 5,104,646, Bolich et al., U.S. Pat. No. 5,618,524, issued Apr. 8, 1997, all of which are incorporated by reference herein in their entirety.

(c). Synthetic Organic Polymers

Also suitable for use as film forming polymers are polymers made by polymerizing one or more ethylenically unsaturated monomers. The final polymer may be a homopolymer, copolymer, terpolymer, or graft or block copolymer, and may contain monomeric units such as acrylic acid, methacrylic acid or their simple esters, styrene, ethylenically unsaturated monomer units such as ethylene, propylene, butylene, etc., vinyl monomers such as vinyl chloride, styrene, and so on.

In some cases, polymers containing one or more monomers which are esters of acrylic acid or methacrylic acid, including aliphatic esters of methacrylic acid like those obtained with the esterification of methacrylic acid or acrylic acid with an aliphatic alcohol of 1 to 30, preferably 2 to 20, more preferably 2 to 8 carbon atoms. If desired, the aliphatic alcohol may have one or more hydroxy groups are particularly suitable. Also suitable are methacrylic acid or acrylic acid esters esterified with moieties containing alicyclic or bicyclic rings such as cyclohexyl or isobornyl, for example.

The ethylenically unsaturated monomer may be mono-, di-, tri-, or polyfunctional as regards the addition-polymerizable ethylenic bonds. A variety of ethylenically unsaturated monomers are suitable.

Examples of suitable monofunctional ethylenically unsaturated monomers include those of the formula:

I.

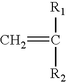

wherein R₁ is H, a C₁₋₃₀ straight or branched chain alkyl, aryl, aralkyl; R₂ is a pyrrolidone, a C₁₋₃₀ straight or branched chain alkyl, or a substituted or unsubstituted aromatic, alicyclic, or bicyclic ring where the substitutents are C₁₋₃₀ straight or branched chain alkyl, or COOM or OCOM wherein M is H, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl groups, or [(CH₂)ₘO]ₙH wherein m is 1-20, and n is 1-200.

More specific examples include the monofunctional ethylenically unsaturated monomer is of Formula I, above, wherein R₁ is H or a C₁₋₃₀ alkyl, and R₂ is COOM or OCOM wherein M is a C₁₋₃₀ straight or branched chain alkyl which may be substituted with one or more hydroxy groups.

Further examples include where R₁ is H or CH₃, and R₂ is COOM wherein M is a C₁₋₁₀ straight or branched chain alkyl which may be substituted with one or more hydroxy groups.

Di-, tri- and polyfunctional monomers, as well as oligomers, of the above monofunctional monomers may also be used to form the polymer. Suitable difunctional monomers include those having the general formula:

II.

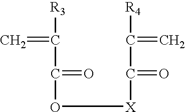

wherein R₃ and R₄ are each independently H, a C₁₋₃₀ straight or branched chain alkyl, aryl, or aralkyl; and X is [(CH₂)ₓO_{y}]_{z} wherein x is 1-20, and y is 1-20, and z is 1-100. Particularly preferred are difunctional acrylates and methacrylates, such as the compound of formula II above wherein R₃ and R₄ are CH₃ and X is [(CH₂)ₓO_{y}]_{z} wherein x is 1-4; and y is 1-6; and z is 1-10.

Trifunctional and polyfunctional monomers are also suitable for use in the polymerizable monomer to form the polymer used in the compositions of the invention. Examples of such monomers include acrylates and methacrylates such as trimethylolpropane trimethacrylate or trimethylolpropane triacrylate.

The polymers can be prepared by conventional free radical polymerization techniques in which the monomer, solvent, and polymerization initiator are charged over a 1-24 hour period of time, preferably 2-8 hours, into a conventional polymerization reactor in which the constituents are heated to about 60-175° C., preferably 80-100° C. The polymers may also be made by emulsion polymerization or suspension polymerization using conventional techniques. Also anionic polymerization or Group Transfer Polymerization (GTP) is another method by which the copolymers used in the invention may be made. GTP is well known in the art and disclosed in U.S. Pat. Nos. 4,414,372; 4,417,034; 4,508,880; 4,524,196; 4,581,428; 4,588,795; 4,598,161; 4,605,716; 4,605,716; 4,622,372; 4,656,233; 4,711,942; 4,681,918; and 4,822,859; all of which are hereby incorporated by reference.

Also suitable are polymers formed from the monomer of Formula I, above, which are cyclized, in particular, cycloalkylacrylate polymers or copolymers having the following general formulas:

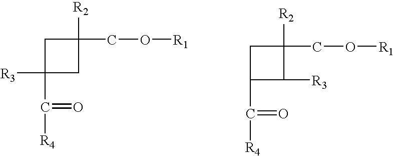

wherein R₁, R₂, R₃, and R₄ are as defined above. Typically such polymers are referred to as cycloalkylacrylate polymers. Such polymers are sold by Phoenix Chemical, Inc. under the tradename Giovarez AC-5099M. Giovarez has the chemical name isododecane acrylates copolymer and the polymer is solubilized in isododecane. The monomers mentioned herein can be polymerized with various types of organic groups such as propylene glycol, isocyanates, amides, etc.

One type of organic group that can be polymerized with the above monomers includes a urethane monomer. Urethanes are generally formed by the reaction of polyhydroxy compounds with diisocyanates, as follows:

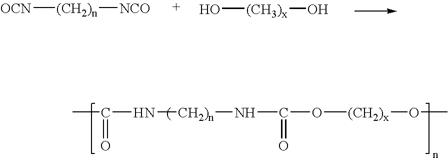

wherein x is 1-1000.

Another type of monomer that may be polymerized with the above comprise amide groups, preferably having the the following formula:

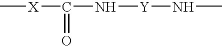

wherein X and Y are each independently linear or branched alkylene having 140 carbon atoms, which may be substituted with one or more amide, hydrogen, alkyl, aryl, or halogen substituents.

Another type of organic monomer may be alpha or beta pinenes, or terpenes, abietic acid, and the like.

One additional type of synthetic organic polymer that may be used in the compositions of the invention is obtained by polymerizing ethylenically unsaturated monomers which comprise vinyl ester groups either alone or in combination with other monomers including silicon monomers, other ethylenically unsaturated monomers, or organic groups such as amides, urethanes, glycols, and the like. The various types of monomers or moieties may be incorporated into the film forming polymer by way of free radical polymerization, addition polymerization, or by formation of grafts and blocks which are attached to the growing polymer chain according to processes known in the art. Preferably the film forming polymer is an organic synthetic polymer obtained by polymerizing ethylenically unsaturated monomers comprised of vinyl ester groups and optionally organic or silicon groups or other types of ethylenically unsaturated monomers.

Typically, the film forming polymer comprises vinyl ester monomers having the following general formula:

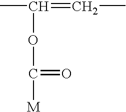

wherein M is H, or a straight or branched chain C₁₋₁₀₀ alkyl, preferably a C₁₋₅₀ alkyl, more preferably a C₁₋₄₅ alkyl which may be saturated or unsaturated, or substituted or unsubstituted, where the substituents include hydroxyl, ethoxy, amide or amine, halogen, alkyloxy, alkyloxycarbonyl, and the like. Preferably, M is H or a straight or branched chain alkyl having from 1 to 30 carbon atoms. The film forming polymer may be a homopolymer or copolymer having the vinyl ester monomers either alone or in combination with other ethylenically unsaturated monomers, organic groups, or silicon monomers.

Suitable other monomers that may be copolymerized with the vinyl ester monomer include those having siloxane groups, including but not limited to those of the formula:

[SiO]ₙ

wherein n ranges from 1-1,000,000. The silicon monomers are preferably polymerized into a siloxane polymer then attached to the polymer chain by attaching a terminal organic group having olefinic unsaturation such as ethylene or propylene, to the siloxane, then reacting the unsaturated group with a suitable reactive site on the polymer to graft the siloxane chain to the polymer.

Also suitable are various types of organic groups that may be polymerized with the vinyl ester monomers including but not limited to urethane, amide, polyalkylene glycols, and the like as set forth above.

The vinyl ester monomers may also be copolymerized with other ethylenically unsaturated monomers that are not vinyl esters, including those set forth above.

(d). Natural Polymers

Also suitable for use are one or more naturally occuring polymeric materials such as resinous plant extracts including such as rosin, shellac, and the like.

Preferred compositions of the invention comprise one or more film forming polymers.

E. Structuring Agents

The compositions of the invention may comprise one more structuring agents. The term “structuring agent” means an ingredient or combination of ingredients that increase the viscosity of, or thicken, the composition. Suggested ranges of structuring agent range from about 0.01-65%, preferably about 0.05-50%, more preferably about 0.1-45% by weight of the total composition.

One type of structuring agent comprises natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like. Particularly preferred is Quaternium-18 hectorite.

Also suitable as structuring agents are various polymeric compounds known in the art as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonyphenyl

Another type of structuring agent that may be used in the compositions are silicas, silicates, silica silylate, and derivatives thereof. These silicas and silicates are generally found in the particulate form. Particularly preferred is silica.

Also suitable as structuring agents are cross-linked organosiloxane compounds also known as silicone elastomers. Such elastomers are generally prepared by reacting a dimethyl methylhydrogen siloxane with a crosslinking group comprised of a siloxane having an alkylene group having terminal olefinic unsaturation or with an organic group having an alpha or omega diene. Examples of suitable silicone elastomers for use as thixotropic agents include Dow Corning 9040, sold by Dow Corning, and various elastomeric silicones sold by Shin-Etsu under the KSG tradename including KSG 15, KSG 16, KSG 19 and so on.

Suitable structuring agents include natural or synthetic waxes. A variety of waxes are suitable including animal, vegetable, mineral, or silicone waxes. Generally such waxes have a melting point ranging from about 28 to 125° C., preferably about 30 to 100° C. Examples of waxes include acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax; or fatty acids or esters such as hydroxystearic acids (for example 12-hydroxy stearic acid), tristearin, tribehenin, and so on.

Also suitable are various types of silicone waxes, referred to as alkyl silicones, which are polymers that comprise repeating dimethylsiloxy units in combination with one or more methyl-long chain alkyl siloxy units wherein the long chain alkyl is generally a fatty chain that provides a wax-like characteristic to the silicone. Such silicones include, but are not limited to stearoxydimethicone, behenoxy dimethicone, stearyl dimethicone, cetearyl dimethicone, and so on. Suitable waxes are set forth in U.S. Pat. No. 5,725,845, which is hereby incorporated by reference in its entirety.

Also suitable as structuring agents are various types of polyamides or silicone polyamides including those set forth in U.S. Patent Publication Nos. 2002/0114773 or 2003/0072730, both of which are hereby incorporated by reference in their entirety.

F. Oils

It may be desirable to include one or more oils in the composition. If so, suggested ranges are from about 0.1-85%, preferably about 0.5-75%, more preferably about 1-70% by weight of the total composition. Such oils are generally liquid at room temperature and are preferably of lower viscosity. If the nonvolatile oils are too heavy or greasy it may hamper the desired long wearing characteristics of the invention. Generally, the viscosity of the nonvolatile oils, if present, may vary depending on the type of composition being formulated, including viscosities ranging from about 2 to 1,000,000 centipoise at 25° C. If lower viscosity oils are desired, a preferred subranges includes from about 11-1000, preferably less than 100 centipoise, most preferably less than about 50 centipoise at 25° C. Examples of such oils include polyalkylsiloxanes, polyarylsiloxanes, and polyethersiloxanes. Examples of such nonvolatile silicones are disclosed in Cosmetics, Science and Technology 27-104 (Balsam and Sagarin ed. 1972); and U.S. Pat. Nos. 4,202,879 and 5,069,897, both of which are hereby incorporated by references. Further nonlimiting examples of such silicones include dimethicone, phenyl trimethicone, trimethylsiloxyphenyldimethicone, dimethicone copolyol, and so on.

Also suitable are lower viscosity organic liquids including saturated or unsaturated, substituted or unsubstituted branched or linear or cyclic organic compounds that are liquid under ambient conditions. Preferred organic liquids include those described in U.S. Pat. Nos. 5,505,937; 5,725,845; 5,019,375; and 6,214,329, all of which are incorporated by reference herein in their entirety. Such silicones or organic oils include those further described as follows:

(a). Esters

Suitable esters are mono-, di-, and triesters. The composition may comprise one or more esters selected from the group, or mixtures thereof.

(i). Monoesters

Monoesters are defined as esters formed by the reaction of a monocarboxylic acid having the formula R—COOH, wherein R is a straight or branched chain saturated or unsaturated alkyl having 2 to 150 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, i.e. may have from about 6 to 30 carbon atoms. Examples of monoester oils that may be used in the compositions of the invention include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, ceyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid.

(ii). Diesters

Suitable diesters that may be used in the compositions of the invention are the reaction product of a dicarboxylic acid and an aliphatic or aromatic alcohol. The dicarboxylic acid may contain from 2 to 150 carbon atoms, and may be in the straight or branched chain, saturated or unsaturated form. The dicarboxylic acid may be subsituted with one or more hydroxyl groups. The aliphatic or aromatic alcohol may also contain 2 to 30 carbon atoms, and may be in the straight or branched chain, saturated, or unsaturated form. The aliphatic or aromatic alcohol may be substituted with one or more substitutents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisostearyl dimer dilinoleate, disostearyl fumarate, diisostearyl malate, and so on.

(iii). Triesters

Suitable triesters comprise the reaction product of a tricarboxylic acid and an aliphatic or aromatic alcohol. As with the mono- and diesters mentioned above, the acid and alcohol contain 2 to 150 carbon atoms, and may be saturated or unsatured, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, triisostearyl citrate, tri C₁₂₋₁₃ alkyl citrate, tricaprylin, tricaprylyl citrate, tridecyl behenate, trioctyldodecyl citrate, trioctydodecyl citrate dilinoleate, tridecyl behenate, tridecyl cocoate, tridecyl isononanoate, and so on.

(b). Hydrocarbon Oils.

It may be desirable to incorporate one or more hydrocarbon oils into the composition of the invention. The hydrocarbons are preferably non-volatile. The term “non-volatile” means that the oil has a vapor pressure of less than about 2 mm. of mercury at 20° C.

Suitable nonvolatile hydrocarbon oils include isoparaffins and olefins, preferably those having greater than 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, mineral oil, pentahydrosqualene, squalene, squalane, and mixtures thereof.

(c). Lanolin Oil

Also suitable for use in the composition is lanolin oil or derivatives thereof containing hydroxyl, alkyl, or acetyl groups, such as hydroxylated lanolin, isobutylated lanolin oil, acetylated lanolin, acetylated lanolin alcohol, and so on.

(d). Glyceryl Esters of Fatty Acids

The nonvolatile oil may also comprise naturally occuring glyceryl esters of fatty acids, or triglycerides. Both vegetable and animal sources may be used. Examples of such oils include castor oil, lanolin oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable are synthetic or semi-synthetic glyceryl esters, e.g. fatty acid mono-, di-, and triglycerides which are natural fats or oils that have been modified, for example, acetylated castor oil, or mono-, di- or triesters of polyols such as glyceryl stearate, diglyceryl diiosostearate, polyglyceryl-4 isostearate, polyglyceryl-6 ricinoleate, glyceryl dioleate, glyceryl diisotearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

(e). Nonvolatile Silicones

Nonvolatile silicone oils, both water soluble and water insoluble, are also suitable for use as the non-volatile oil. Such silicones preferably have a viscosity ranging from about 10 to 600,000 centipoise, preferably 20 to 100,000 centipoise at 25° C. Suitable water insoluble silicones include amine functional silicones such as amodimethicone; phenyl substituted silicones such as bisphenylhexamethicone, phenyl trimethicone, or polyphenylmethylsiloxane; dimethicone, alkyl substituted dimethicones, and mixtures thereof.

Water soluble, non-film forming silicones such as dimethicone copolyol, dimethiconol, and the like may be used. Such silicones are available from Dow Corning as the 3225C formulation aid, Dow 190 and 193 fluids, or similar products marketed by Goldschmidt under the ABIL tradename.

(f). Fluorinated Oils

Also suitable as the oil are various fluorinated oils such as fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers like those disclosed in U.S. Pat. Nos. 5,183,589,4,803,067, 5,183,588 all of which are hereby incorporated by reference, which are commercially available from Montefluos under the trademark Fomblin.

Fluoroguerbet esters are also suitable oils. The term “guerbet ester” means an ester which is formed by the reaction of a guerbet alcohol having the general formula:

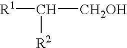

and a fluoroalcohol having the following general formula:

CF₃—(CF₂)ₙ—CH₂—CH₂—OH

wherein n is from 3 to 40.

with a carboxylic acid having the general formula:

R³COOH, or

HOOC—R³—COOH

wherein R¹, R², and R³ are each independently a straight or branched chain alkyl.

Preferably, the guerbet ester is a fluoro-guerbet ester which is formed by the reaction of a guerbet alcohol and carboxylic acid (as defined above), and a fluoroalcohol having the following general formula:

ti CF₃—(CF₂)ₙ—CH₂—CH₂—OH

wherein n is from 3 to 40.

Examples of suitable fluoro guerbet esters are set forth in U.S. Pat. No. 5,488,121 which is hereby incorporated by reference. Suitable fluoro-guerbet esters are also set forth in U.S. Pat. No. 5,312,968 which is hereby incorporated by reference. One type of such an ester is fluorooctyldodecyl meadowfoamate, sold under the tradename Silube GME-F by Siltech, Norcross Ga.

The compositions may contain one or more additional ingredients including preservatives, humectants, antioxidants, and the like.

In another embodiment, the cosmetic compositions of the invention comprise a pigmented cosmetic composition comprising as an ingredient at least one organic solvent containing at least one short chain alkyl ether group bonded to at least one short chain alcohol or alcohol ester. The composition may be an eyeshadow, blush, lipstick, concealer, foundation, eyeliner, mascara, and the like, and contain the ingredients and in the ranges set forth herein.

The invention will be further described in connection with the following examples which are set forth for the purposes of illustration only.

### EXAMPLE 1

A lip color composition where a portion of the volatile solvent was replaced with methoxypropanol is prepared as follows:

| Ingredient | % by Wt. |
|---|---|
| Trimethylsiloxysilicate | 45.00 |
| Isododecane | 32.70 |
| Methoxypropanol | 4.50 |
| Trimethylsiloxy phenyldimethicone | 11.0 |
| Quaternium-18 hecorite | 0.50 |
| Propylene carbonate | 0.10 |
| 12-Hydroxystearic acid | 0.20 |
| Pigment/Pearls/Mica | 6.00 |

The composition is prepared by grinding the pigments/pearls/mica in a portion of the isododecane. Separately, the remaining ingredients are combined and mixed well, and the pigment grind added. The resulting composition is a viscous semi-solid.

### EXAMPLE 2

Another lip color composition where a portion of the volatile silicone and volatile paraffinic hydrocarbon are replaced with methoxypropanol is prepared as follows:

| Ingredient | % by Wt. |
|---|---|
| Polyvinyl neononanoate | 34.5 |
| Dimethicone - 1 centistoke viscosity | 37.0 |
| Isododecane/quaternium-18 hectorite | 15.0 |
| propylene carbonate | |
| methoxypropanol | 3.5 |
| Pigments/Pearls/Mica | 10.0 |

The composition is prepared by grinding the pigments/pearls/mica in a portion of the dimethicone. The rest of the ingredients are combined and mixed well, the pigment grind added, and the ingredients mixed well. The resulting composition is a viscous semi-solid.

### EXAMPLE 3

Other lipcolor compositions where a portion of the isododecane is replaced with methoxypropanol are set forth below:

| | 1 | 2 | 3 |
|---|---|---|---|
| Polysilicone 6 | 21 | — | — |
| Trimethylsiloxysilicate | — | 40.00 | 33.00 |
| Isododecane | 60 | 38.00 | 45.00 |
| Methoxypropanol | 3.5 | 6.00 | 4.00 |
| Tocopheryl acetate | 0.2 | — | — |
| Aloe | 0.1 | — | — |
| Methyl paraben | 0.3 | — | — |
| BHA | 0.1 | — | — |
| Color/pearl/mica | 14.00 | 8.00 | 13.00 |
| Trioctyldodecyl citrate | 0.80 | — | — |
| Octylmethoxycinnamate | — | — | 3.00 |
| Dimethicone (100,000 cst.) | — | 4.00 | — |
| Methicone treated mica | — | 2.00 | — |
| Methicone treated iron oxide | — | — | 2.00 |
| Bismuth oxychloride | — | 1.00 | — |
| Octyl isononanoate | — | 1.00 | — |

The compositions were made by grinding the pigments in a portion of the oily ingredients. Then, the remaining ingredients were combined and mixed to form the final composition.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. In a long wearing and/or transfer resistant cosmetic composition wherein at least a portion of the long wearing or transfer resistant property is provided by volatile silicones or volatile paraffinic hydrocarbons, the improvement wherein at least some of the volatile silicone or volatile paraffinic hydrocarbon is replaced with an organic solvent containing at least one short chain alkyl ether group bonded to at least one short chain alcohol or alcohol ester wherein said substitution enables the composition to retain its long wearing and/or transfer resistant character.

2. The composition of claim 1 wherein the organic solvent has a viscosity of less than about 8 centipoise at 25° C.

3. The composition of claim 1 wherein the organic solvent has a vapor pressure of at least about 2 mm. of mercury at 20° C.

4. The composition of claim 1 wherein the organic solvent has a vapor pressure ranging from about 1.0 to 2.0 mm. of mercury at 20° C.

5. The composition of claim 1 wherein the organic solvent has a vapor pressure ranging from about 0.5 to 20 mm. of mercury at 20° C.

6. The composition of claim 1 wherein the at least one short chain alkyl ether group is a C₁, C₂, C₃, C₄, C₅, or C₆ alkyl ether.

7. The composition of claim 1 wherein the at least one short chain alkyl ether group is a C₁, C₂, C₃, or C₄ alkyl ether.

8. The composition of claim 1 wherein the at least one short chain alkyl ether group is a C₁, C₂, or C₃ alkyl ether.

9. The composition of claim 1 wherein the at least one short chain alkyl ether group is methoxy or ethoxy.

10. The composition of claim 1 wherein the at least one short chain alcohol is a C₁, C₂, C₃, C₄, C₅, or C₆ mono-, di-, or polyhydric alcohol.

11. The composition of claim 1 wherein the at least one short chain alcohol is a C₁, C₂, C₃, C₄, C₅, or C₆ monohydric alcohol.

12. The composition of claim 1 wherein the at least one short chain alcohol is a C₁ C₂, C₃, C₄, C₅, or C₆ dihydric alcohol.

13. The composition of claim 1 wherein the at least one short chain alcohol is a C₁, C₂, C₃, C₄, C₅, or C₆ polyhydric alcohol.

14. The composition of claim 1 wherein the at least one short chain alcohol is a C₁, C₂, C₃, or C₄ monohydric alcohol.

15. The composition of claim 1 wherein the at least one short chain alcohol is a C₁, C₂, or C₃ monohydric alcohol.

16. The composition of claim 1 wherein the at least one short chain alcohol is a C₁ or C₂ monohydric alcohol.

17. The composition of claim 1 wherein the at least one short chain alcohol ester is a C₁₋₆ alcohol esterified with a C₁₋₆ carboxylic acid.

18. The composition of claim 1 wherein the at least one short chain alcohol ester is a C₁₋₄ alcohol esterified with a C₁₋₄ carboxylic acid.

19. The composition of claim 1 wherein the at least one short chain alcohol ester is a C₁₋₃ alcohol esterified with a C₁₋₃ carboxylic acid.

20. The composition of claim 1 wherein the organic solvent s selected from the group consisting of propylene glycol ethyl ether acetate, propylene glycol butyl ether, propylene glycol methyl ether acetate, propylene glycol methyl ether, propylene glycol ethyl ether, and mixtures thereof.

21. The composition of claim 1 further comprising at least one structuring agent.

22. The composition of claim 1 wherein the structuring agent is a natural or synthetic wax.

23. The composition of claim 1 wherein the composition comprises from about 0.01-65% by weight of the total composition of one or more structuring agents.

24. The composition of claim 21 wherein the structuring agent comprises a natural of synthetic montmorillonite mineral.

25. The composition of claim 21 wherein the structuring agent comprises an associative thickener, a silica or silicate, a silicone elastomer, a polyamide or silicone polyamide, or natural or synthetic wax.

26. The composition of claim 1 further comprising an oil.

27. The composition of claim 26 wherein the oil is present at about 0.1-85% by weight of the total composition.

27. The composition of claim 26 wherein the oil comprises a silicone, an ester, a hydrocarbon, lanolin, or mixtures thereof.

28. A method for improving the wear, feel, application, and other aesthetic properties of long wearing cosmetic compositions wherein the long wearing property is achieved, at least in part, by use of volatile silicones or volatile paraffinic hydrocarbons, by substituting at least a portion of the volatile silicone or volatile paraffinic hydrocarbon with an organic solvent containing at least alkyl ether group bonded to at least one short chain alcohol or alcohol ester.

29. The method of claim 28 wherein about 1 to 50% of the volatile silicone or volatile paraffinic hydrocarbon is substituted with the organic solvent.

30. The method of claim 28 wherein all of the volatile silicone or volatile paraffinic hydrocarbon present is substituted with the organic solvent.

31. The method of claim 28 wherein the organic solvent is selected from the group consisting of propylene glycol ethyl ether acetate, propylene glycol butyl ether, propylene glycol methyl ether acetate, propylene glycol methyl ether, propylene glycol ethyl ether, methoxypropanol, and mixtures thereof.

32. A cosmetic composition comprising at least one organic solvent containing at least alkyl ether group bonded to at least one short chain alcohol or alcohol ester.

33. The composition of claim 32 which comprise a pigment.

34. The composition of claim 33 which is a lip color.

35. The composition of claim 34 comprising about 0.05-70% by weight of the total composition of pigments or powders.

36. The composition of claim 35 further comprising about 0.01-90% by weight of the total composition of one or more film forming polymers.

37. The composition of claim 36 which is a lipstick.

38. The composition of claim 36 which is a foundation, concealer, blush, mascara, or eyeshadow.

39. The composition of claim 32 comprising about 0.05-70% by weight of pigments or powders, about 0.01-90% by weight of one or more film forming polymers, about 0.01-65% of one or more structuring agents, and about 0.1-85% of one or more oils.

40. The composition of claim 39 which is a lipstick.
